(19) United States
(12) Patent Application Publication
(10) Pub. No.: US 2008/0306348 A1
(43) Pub. Date: Dec. 11, 2008

(54) MINIATURE WIRELESS APPARATUS FOR RECORDING PHYSIOLOGICAL SIGNALS OF HUMANS AND USE THEREOF

(75) Inventors: Terry B.J. Kuo, Taipei City (TW); Cheryl C.H. Yang, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 11/758,921

(22) Filed: Jun. 6, 2007

(51) Int. Cl. A61B 5/00

(52) U.S. Cl. 600/300

(57) ABSTRACT

A miniature wireless apparatus for recording and remote monitoring physiological signals and use thereof are described. The miniature wireless apparatus comprises a signal collecting module, a signal processing module, a wireless module, and a recording device. The methods for recording and remote monitoring physiological signals comprising collecting physiological signals by a signal collecting module, processing the said physiological signals by a signal processing module and recording the processed signals by a recording device, modulating the said processed signals and synchronously transmitting and receiving the modulated signals by a wireless module to integrate the physiological signals, receiving the signals by a remote receiver, and recording, monitoring, and analyzing the physiological signals by the remote receiver and further transmitting the physiological signals to the remote monitoring station through a network. Methods for sleep monitoring and evaluating and for monitoring and evaluating of autonomic nerve function using the miniature wireless apparatus are further described.

### FIELD OF THE INVENTION

The present invention is related to an apparatus and methods for recording physiological signals, and more specifically to a miniature wireless apparatus for recording and remote monitoring physiological signals and the use thereof.

### BACKGROUND OF THE INVENTION

Physiological signals include heartbeat, electroencephalogram (EEG), breath, body temperature, and so on. All these physiological signals are signs of health, and long-term monitoring of these biological parameters is important to some disease such as heart disease and sleep disorders. Moreover, emergency medical care and intensive care can all benefit from continuous vital sign monitoring, especially immediate notification of patient deterioration. If the related information of the physiological signals can be obtained easily, it benefits to take care of patients, the labor cost can be reduced and the quality of medical care can be improved.

Conventionally, the apparatuses for collecting physiological signals are awkward and expensive. A lot of electrical wires must be connected to the subject and further to the monitor processing stations. Those sensors and their trailing wires severely constrain subject mobility and make the subject uncomfortable. A lot of wires also make it difficult to perform any tests on the patient which require the patient move, and in some cases, the wires can easily become detached from the subject. The inconveniences mentioned above disincline subjects from accepting the long-term monitoring and influence the accuracy of the examination. Along with the progress of science and technology, semiconductor and wireless transmission technologies have become well developed, and the miniature products for detecting physiological signals have been proposed successively. A so-called ambulatory physiological signal-collecting apparatus has been achieved that is the size of a palm. Some of the apparatuses can continuously store the physiological signals in a portable processor. Other apparatuses can transmit the physiological signals to a remote receiver and processor in the form of wireless wave or the IR transmission and further to the nurses' station for real-time monitor. However, electrical wires are still needed to connect sensors and the local processor. Although the electrical wires are short, it is also inconvenient for users to recording a plurality of physiological signals, and the wires is one source of noise. Further, some of the instruments are still too large and cumbersome, and the manner of connecting the wire is too complex for persons who have no training.

Recently, a number of wireless medical monitors are developed. US 20050261559, US 20050249037, and US 20060066449 disclosed the wireless physiological monitoring system wherein physiological signals from the detector are transmitted to the local processor by a wireless transmitter. There are no more wires between sensors and the monitoring device. Also, a number of wearable systems have been proposed with integrated wireless transmission, GPS (Global Positioning System) sensor, and local processing. Commercial systems are also becoming available. The technologies allow the application of the physiological signal detection to be more convenient and flexible. However, the physiological signals are processed and recorded after being transmitted to the monitoring device. Thus, there is a risk of loosing data when the wireless signal is interfered such as in the hospital. Lacking of multi-sensor data integration is also a disadvantage for further analysis of physiological activity. Moreover, the subject must wear more than one apparatus to record a plurality of physical signals. It is still inconvenient to the subject.

In order to spread the physiological signal analysis technology widely to each family and person, it is necessary to overcome the inconvenience of various fixed or portable physiological instruments, and miniaturization and completely wireless instruments inevitably become the direction to follow in development.

### SUMMARY OF THE INVENTION

In accordance with the present invention, a miniature wireless apparatus and methods for recording and remote monitoring physiological signals and use thereof are described. The miniature wireless apparatus for recording and remote monitoring physiological signals comprises a signal collecting module for collecting one or more physiological signals, a signal processing module coupled to the signal collecting module for processing the said physiological signals, a wireless module for modulating the signals from the signal processing module, transmitting the modulated signals, and receiving external wireless signals, and a recording device.

The methods for recording and remote monitoring physiological signals comprise collecting one or more physiological signals by a signal collecting module, processing the said physiological signals by a signal processing module and recording the processed signals by a recording device, modulating the said processed signals and synchronously transmitting and receiving the modulated signals from different miniature wireless apparatuses by a wireless module to integrate the physiological signals, receiving the said modulated signals by a remote receiver, and recording, monitoring, and analyzing the physiological signals by a microcomputer system of the remote receiver and further transmitting the physiological signals to remote monitoring stations through a network.

The miniature wireless apparatus and methods for recording and remote monitoring physiological signals can be further applied to medical uses comprising evaluation of sleep quality, evaluation of diseases of circulation system, diagnosis of sleep disorders, evaluation of effect of hypnotics, evaluation of sleep-related side effects of drugs, evaluation of influences of various regimen on sleep, evaluation of influences of various health food on sleep, evaluation of side effects of drugs on autonomic nerve function, evaluation of influences of various regimen on autonomic nerve function, evaluation of influences of various health food on autonomic nerve function, evaluation of health of the elders, and newborn sleep-disorders screen.

In accordance with the present invention, methods for sleep monitoring and evaluating and for monitoring and evaluating of autonomic nerve function using the miniature wireless apparatus are further described. The method for sleep monitoring and evaluating using the miniature wireless apparatus comprises collecting, processing, and recording a plurality of physiological signals, processing and modulating the said physiological signals to form modulated radio frequency signals, synchronously transmitting and receiving the modulated signals radio frequency signals corresponding to different physiological signals, receiving and recording the physiological signals by a remote receiver and further transferring the signals to remote monitoring stations through a network, and analyzing the physiological signals with a sleep-analyzing algorithm for sleep monitoring and evaluating by the remote receiver and the remote monitoring station.

The method for monitoring and evaluating of autonomic nerve function using the miniature wireless apparatus comprises collecting, processing, and recording a plurality of physiological signals, processing and modulating the said physiological signals to form modulated radio frequency signals, synchronously transmitting and receiving the modulated signals radio frequency signals corresponding to different physiological signals, receiving and recording the physiological signals by a remote receiver and further transferring the signals to remote monitoring stations through a network, and analyzing the physiological signals with an autonomic nervous-analyzing algorithm for monitoring and evaluating of autonomic nerve function by the remote receiver and the remote monitoring station.

The present invention discloses a miniature wireless apparatus and methods for long-term recording physiological signals and for remote and real-time monitoring a plurality of biological parameters. An advantage of the present invention is that an internal receiver, an internal memory, and a processor are incorporated into the detecting apparatus without additional electrical wires or local processor to receive and record biological signals. Another advantage is that different types of sensors can be integrated in one apparatus for more accurate analysis of physiological activity and for convenience. Yet another advantage is that an internal memory incorporated into the detecting apparatus for long-term recording can overcome the predicament of data loosing when the wireless signal is interfered. A further advantage is that synchronous control of signal transmitting can minimize the bandwidth usage. With its low power and low cost design, the miniature wireless apparatus for long-term recording and remote monitoring physiological signals is convenient to users and beneficial to broad application.

These and other exemplary embodiments, features, aspects, and advantages of the present invention will be described and become more apparent from the detailed description of exemplary embodiments when reading in conjunction with accompanying drawings.

### BRIEF DESCRIPTION OF THE DRAWINGS

To have better understanding of the invention and to show more clearly how it may be carried into effect, reference will now be made, by way of example only, to the accompanying drawings which show exemplary embodiments of the invention and in which:

FIG. 1 schematically illustrates a functional block diagram of a miniature wireless apparatus for recording and remote monitoring physiological signals in accordance with the present invention.

FIG. 2 schematically illustrates a miniature wireless apparatus incorporated with a temperature sensor and an acceleration sensor for recording and remote monitoring physiological signals according to an embodiment of the present invention. FIG. 3A schematically illustrates methods for recording and remote monitoring ECG signals using the miniature wireless apparatus described schematically in FIG. 2 in accordance with the present invention.

FIG. 3B schematically illustrates methods for recording and remote monitoring EGG signals using the miniature wireless apparatus described schematically in FIG. 2 in accordance with the present invention.

FIG. 3C schematically illustrates methods for recording and remote monitoring a plurality of physiological signals using the miniature wireless apparatus described schematically in FIG. 2 in accordance with the present invention.

FIG. 4 schematically illustrates methods for synchronously transmitting and receiving the physiological signals by dividing the receiving-time of signals from different apparatus in accordance with the present invention.

### DETAILED DESCRIPTION OF THE PREFERRED EMBODIMENTS

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements. In addition, numerous specific details are set forth in order to provide a thorough understanding of the invention. However, it will be understood by those of ordinary skill in the art that the invention may be practiced without these specific details. In other instances, well-known methods, procedures and components have not been described in detail so as not to obscure the invention.

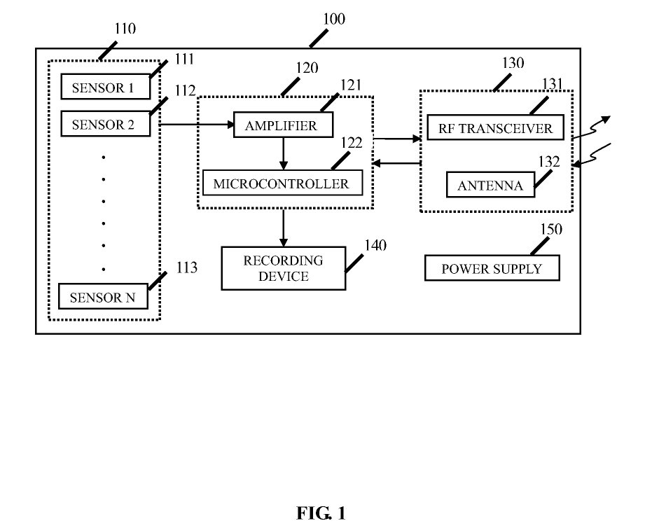

Referring to FIG. 1, shown therein is an exemplary embodiment of a miniature wireless apparatus 100 for recording and remote monitoring physiological signals described in schematic form. The miniature wireless apparatus comprises a signal collecting module 110, a signal processing module 120 coupled to the signal collecting module, a wireless module 130, a recording device 140, and a power supply 150. The signal collecting module 110 comprises at least one wireless or non-wireless sensor for collecting one or more physiological signals. Those skilled in the art can understand that the so-called non-wireless sensor refers to the sensor collecting physiological signals without additional electrical wires. Typically, there are one or more sensors 111, 112, 113, each measuring their respective physiological signal from a subject. Examples of physiological signals include an electroencephalogram (EEG) signal, an electrooculogram (EOG) signal, an electrocardiogram (ECG) signal, an electromyogram (EMG) signal, an electrogastrography (EGG) signal, a temperature signal, and an acceleration signal. The measured physiological signals are then transferred to the signal processing module 120. The signal processing module 120 comprises an amplifier 121 for amplifying the electrical physiological signals and a microcontroller 122 for performing an analog-to-digital conversion and data compression for an amplified physiological signal generated by the amplifier. The amplifier 121 is an operational amplifier for processing single-point signals or differential signals. Those who are familiar with the art will understand that the microcontroller 122 includes all functions that have come to be associated with highly integrated programmable digital devices. The microcontroller 122 selects the signals individually, converts the signals to a digital form, and produces the digital output signals which are then transferred to a recording device 140 and a wireless module 130. The microcontroller 122 further controls the operation of the miniature wireless apparatus 100. The recording device 140 receives the signals from the microcontroller 122 and stores the physiological signals. The recording device 140 may be any suitable memory device, such as a hard disk, a floppy disc, a tape, or a nonvolatile memory. Preferably, the recording device 140 is a nonvolatile memory. More preferably, the recording device 140 is a flash memory.

In one exemplary embodiment, the wireless module 130 comprises an RF transceiver 131 and an antenna 132. The RF (radio frequency) transceiver 131 receives the signals from the microcontroller 122 and modulates the digital signals to form modulated radio frequency (RF) signals which are then transferred to an antenna 132 to generate radiating electromagnetic signals that are received by receivers. The wireless module 130 performs wireless transmission and receiving using an industry, science, and medical (ISM) exclusive frequency band. The wireless module 130 not only transmits the modulated radio frequency signals but also receives the modulated radio frequency signals from the miniature wireless apparatus itself and from other miniature wireless apparatuses on the body of a user when other apparatuses are used simultaneously. For this purpose, an internal receiver is included in the wireless module 130. The physiological signals received by the internal receiver are demodulated and transferred to the signal processing module 120 for producing digital signals and for decoding, and then further stored by the recording device 140 for long-term recording. For integration of physiological signals from different apparatuses used simultaneously, the wireless module 130 further comprises a firmware for synchronously transmitting and receiving the physiological signals from different apparatuses. The wireless module 130 also receives wireless signals of remote manipulation of the apparatus. Electrical power for the miniature wireless apparatus 100 is provided by the power supply 150 for mobile portable operation.

With the progress of semiconductor technology, all the components of the miniature wireless apparatus can be further integrated into one circuit board, or even one chip. The miniature wireless apparatus 100 for recording and remote monitoring physiological signals can further comprise an outer waterproof film or an outer waterproof cover, such that the subject under monitoring feels free in doing various activities. The miniature wireless apparatus 100 is further applied to medical uses including real-time monitoring of vital signs, patient monitoring in hospital, and home health monitoring. The popularization of the methods for recording and remote monitoring of physiological signals is also helpful in prevention, monitoring, and diagnosis of diseases.

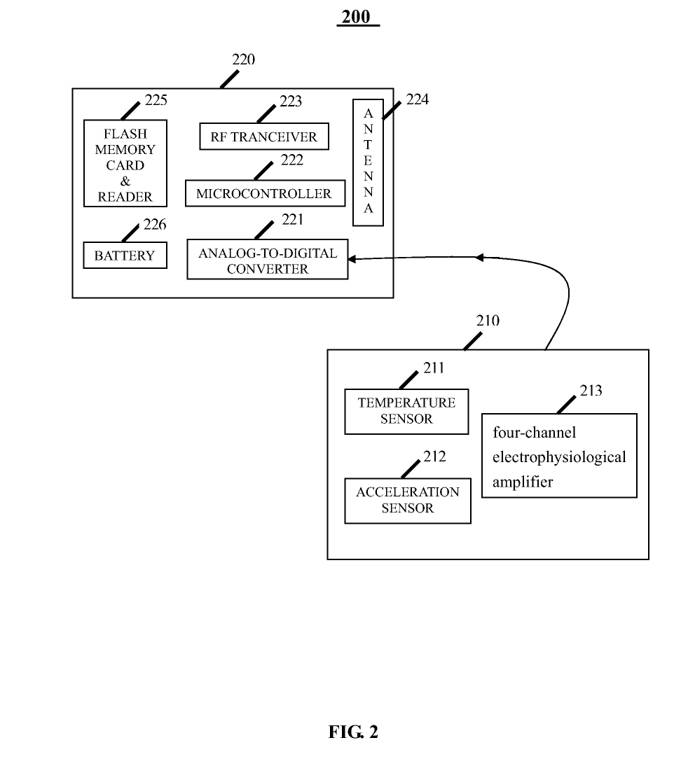

Referring to FIG. 2, shown therein is an exemplary embodiment of a miniature wireless apparatus 200 incorporated with a temperature sensor 211 and an acceleration sensor 212 for recording and remote monitoring physiological signals described in schematic form. The miniature wireless apparatus comprises a temperature sensor 211, an acceleration sensor 212, and a four-channel electrophysiological amplifier 213 on one side 210 of the apparatus 200, and an analog-to-digital converter 221, a microcontroller 222, an RF transceiver 223, an antenna 224, a recording device 225, and a battery 226 on the opposite side 220 of the apparatus. The temperature sensor 211 detecting the body temperature comprises a thermal resistor and an operational amplifier for converting a temperature into a voltage signal. The acceleration sensor 212 is a miniature motion sensor for detecting 3-dimensional acceleration of body motion and the signals are converted to analog voltage signals. The measured signals from the sensors 211, 212 are transferred to the four-channel electrophysiological amplifier 213 for amplifying the electrical physiological signals which are then transferred to an analog-to-digital converter 221. The analog-to-digital converter 221 performs an analog-to-digital conversion and data compression for amplified physiological signals generated by the amplifier 213. The digital output signals from the analog-to-digital converter 221 are then transferred to a recording device 225 and an RF (radio frequency) transceiver 223.

The RF (radio frequency) transceiver 223 receives the signals from the analog-to-digital converter 221 and modulates the digital signals to form modulated radio frequency (RF) signals which are then transferred to an antenna 224 to generate radiating electromagnetic signals that are received by receivers. The RF transceiver 223 and the antenna 224 not only transmit the modulated radio frequency signals but also receive the modulated radio frequency signals from the miniature wireless apparatus itself and from other miniature wireless apparatuses on the body of a user when other apparatuses are used simultaneously. For this purpose, an internal receiver is included in the RF transceiver 223. The physiological signals received by the internal receiver are demodulated and transferred to the analog-to-digital converter 221, and then further stored by the recording device 225 for long-term recording. For integration of physiological signals from different apparatuses used simultaneously, the RF transceiver 223 further comprises a firmware for synchronously transmitting and receiving the physiological signals from different apparatuses. The RF transceiver 223 also receives wireless signals of remote manipulation of the apparatus and couples the signals to the analog-to-digital converter 221 and further to the microcontroller 222. In one embodiment, the RF transceiver is an RFIC (radio frequency integrated chip) with the carrier frequency of 2.4 GHz.

The recording device 225 receives the signals from the analog-to-digital converter 221 and stores the physiological signals collected from other miniature wireless apparatuses used simultaneously on the body of a user. In one embodiment, the recording device 225 comprises a flash memory card and a reader. The flash memory card is a form of nonvolatile memory which provides the convenience of data access with capacity from 256 MB to 4 GB. Along with the progress of electrical science and technology, the flash memory card with higher capacity will be more beneficial to long-term recording. Electrical power for the miniature wireless apparatus 200 is provided by the battery 226 for mobile portable operation. In one embodiment, the battery 226 is a rechargeable lithium battery. The miniature wireless apparatus 200 is guided by remote manipulation and further comprises a push-button switch for emergency shut-down (not shown). The miniature wireless apparatus 200 also comprises an alarm (not shown).

With its low power, low cost, and convenient design, the miniature wireless apparatus is beneficial to long-tern recording and remote monitoring physiological signals and to broad application of a remote monitoring system.

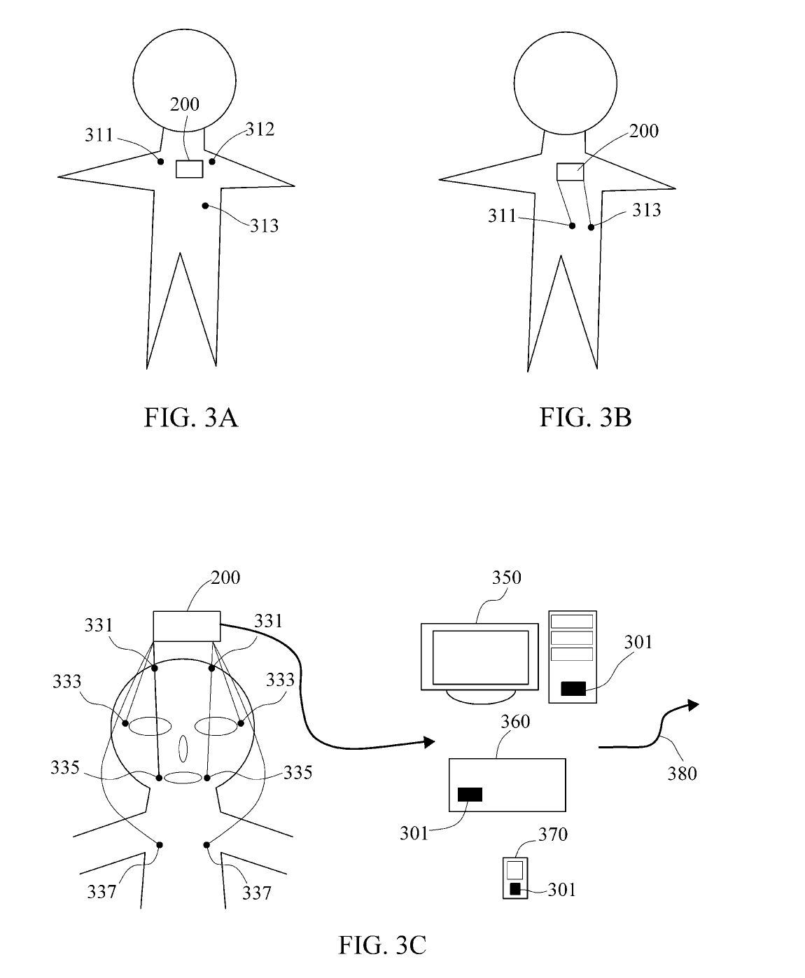

Referring to FIG. 3A, shown therein is an exemplary embodiment of methods for recording and remote monitoring ECG (electrocardiogram) signals using the miniature wireless apparatus 200 described schematically in FIG. 2. Electrodes 311, 312, 313 attached to a subject to collect ECG signals are differential. Those skilled in the art can understand that the standard leads of ECG signals are composed of two electrodes, one being negative and the other one being positive, and the difference in electrical potential between them is recorded, in which: lead I is composed of the electrode 311 on the right arm designated as negative shown in FIG. 3A, and the electrode 312 on the left arm considered positive; and lead II is composed of electrode 311 on the right arm, which is made negative, and the electrode 313 on the left leg, which is considered positive. However, it is not limited to such an approach.

Referring to FIG. 3B, shown therein is an exemplary embodiment of methods for recording and remote monitoring EGG signals using the miniature wireless apparatus 200 described schematically in FIG. 2. The electrode pair 321 is differential and comprises a positive and a negative electrode attached to the subject to collect a pair of EGG (electrogastrography) signals.

Referring to FIG. 3C, shown therein is an exemplary embodiment of methods for recording and remote monitoring a plurality of physiological signals using the miniature wireless apparatus 200 described schematically in FIG. 2. The physiological signals include a temperature signal, an acceleration signal, an EEG signal, an EOG signal, an EMG signal, and an ECG signal. The electrode pair 331, 333, 335, 337 are attached to the subject to collect a pair of EEG (electroencephalogram) signals, EOG (electrooculogram) signals, EMG (electromyogram) signal, and ECG (electrocardiogram) signals, respectively. The physiological signals collected from the apparatus 200 are recorded by the recording device 225 described schematically in FIG. 2 and transmitted to a remote receiver 301 and an internal receiver of the apparatus 200. The remote receiver 301 is included in a microcomputer system such as a personal computer 350, a radio station (not shown), a wireless base station (not shown), a mobile phone 370, and a portable microcomputer system such as a PDA (personal digital assistant) 360. The physiological signals received by the remote receiver are recorded, monitored, and analyzed by the microcomputer system and further transferred to remote monitoring stations through the network 380. The remote monitoring station can be one or more fixed or portable monitoring station, such as a computer in a hospital, a mobile phone of a supervisor, and the like. Those who are familiar with the art will understand that the network 380 includes any wireless and non-wireless network such as GPRS (General Packet Radio Service) and LAN (Local Area Network). The remote monitoring stations carry out storage, analysis, display, and control functions.

The physiological signals can be analyzed with a sleep-analyzing algorithm for sleep monitoring and evaluating, and with an autonomic nervous-analyzing algorithm for autonomic nerve function by the remote receiver and the remote monitoring station. Results of analysis can be applied to medical uses comprising evaluation of sleep quality, evaluation of diseases of circulation system, diagnosis of sleep disorders, evaluation of effect of hypnotics, evaluation of sleep-related side effects of drugs, evaluation of influences of various regimen on sleep, evaluation of influences of various health food on sleep, evaluation of side effects of drugs on autonomic nerve function, evaluation of influences of various regimen on autonomic nerve function, evaluation of influences of various health food on autonomic nerve function, evaluation of health of the elders, and newborn sleep-disorders screen.

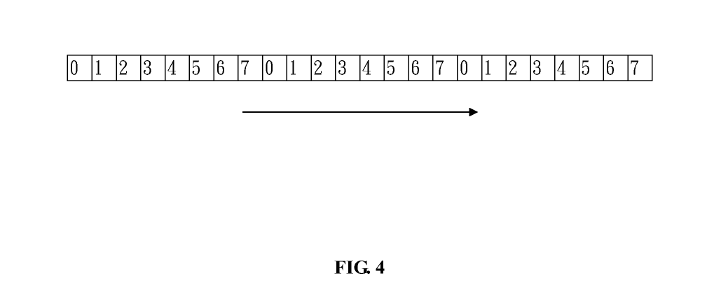

Referring to FIG. 4, shown therein is an exemplary embodiment of methods for synchronously transmitting and receiving the physiological signals by dividing the receiving-time of signals from different apparatuses. As was stated earlier, the wireless module 130 further comprises a firmware for synchronously transmitting and receiving the physiological signals to integrate a plurality of physiological signals from different apparatuses used simultaneously. With the firmware, the wireless module 130 receives the signals, divides the receiving-time into “n” equal intervals, and then corresponds each divided time-interval to a signal which is detected by a particular apparatus. In one embodiment, “n” is ranged from 1 to 50. In the preferred embodiment, “n” is ranged from 1 to 30. In a more preferred embodiment, “n” is ranged from 1 to 10.

Although specific embodiments have been illustrated and described herein for purposes of description of the preferred embodiment, it will be appreciated by those of ordinary skill in the art that a wide variety of alternate and/or equivalent implementations calculated to achieve the same purposes may be substituted for the specific embodiments shown and described without departing from the scope of the present invention. Various modifications and substitutions for the illustrative devices, interactive video technology, and wireless communication system; and for the computer hardware and software technology may be made by those skilled in the relevant art without departing from the novel spirit and scope of the present invention. Therefore, it is manifestly intended that this invention be limited only by the claims and the equivalents thereof.

What is claimed is:

1. A miniature wireless apparatus for recording and remote monitoring physiological signals, comprising:
(a) a signal collecting module for collecting one or more physiological signals;
(b) a signal processing module coupled to the signal collecting module for processing the said physiological signals;
(c) a wireless module for modulating the signals from the signal processing module, transmitting the modulated signals, and receiving external wireless signals; and
(d) a recording device.

2. The miniature wireless apparatus of claim 1, wherein the signal collecting module comprises one or more sensors for detecting physiological signals.

3. The miniature wireless apparatus of claim 2, wherein the sensors comprises wireless or non-wireless sensors.

4. The miniature wireless apparatus of claim 3, wherein the sensors comprises an electroencephalogram sensor, an electrooculogram sensor, an electrocardiogram sensor, an electromyogram sensor, an electrogastrography sensor, an temperature sensor, and an acceleration sensor.

5. The miniature wireless apparatus of claim 4, wherein the acceleration sensor is a miniature motion sensor for detecting 3-dimensional acceleration and the measured signals of acceleration of motion are converted to analog voltage signals.

6. The miniature wireless apparatus of claim 1, wherein the signal processing module comprises an amplifier for amplifying the electrical physiological signals and a microcontroller for performing analog-to-digital conversion and data compression for an amplified physiological signal generated by the amplifier.

7. The miniature wireless apparatus of claim 6, wherein the amplifier is an operational amplifier for processing single-point signals or differential signals.

8. The miniature wireless apparatus of claim 1, wherein the wireless module comprises an RF transceiver and an antenna for modulating the digital signals from the signal processing module to form modulated radio frequency signals, transmitting the modulated radio frequency signals to an internal and a remote receiver, and receiving external wireless signals.

9. The miniature wireless apparatus of claim 8, wherein the internal receiver comprises the receiver of the apparatus transmitting the signals and a receiver of other miniature wireless apparatuses used simultaneously on the body of a user.

10. The miniature wireless apparatus of claim 8, wherein the remote receiver is a fixed or portable microcomputer system.

11. The miniature wireless apparatus of claim 10, wherein the physiological signals received by the remote receiver are recorded, monitored, and analyzed by the microcomputer system and further transferred to remote monitoring stations through a network.

12. The miniature wireless apparatus of claim 8, wherein the external wireless signals comprises the signals of remote manipulation of the apparatus and the physiological signals from other miniature wireless apparatuses used simultaneously on the body of the user.

13. The miniature wireless apparatus of claim 8, wherein the wireless module further comprises a firmware for synchronously transmitting and receiving the physiological signals from different miniature wireless apparatuses to integrate the physiological signals.

14. The miniature wireless apparatus of claim 13, wherein the firmware divides the signal receiving time into equal intervals and corresponds each divided time-interval to each signal which is detected by a particular apparatus.

15. The miniature wireless apparatus of claim 1, wherein the recording device records the physiological signals colleted from sensors of the apparatus and from sensors of the said other miniature wireless apparatuses used simultaneously on the body of the user.

16. The miniature wireless apparatus of claim 15, wherein the recording device is a hard disk, a floppy disc, a tape, or a nonvolatile memory.

17. The miniature wireless apparatus of claim 1, which further comprises a power supply.

18. The miniature wireless apparatus of claim 1, which is further applied to medical uses comprising evaluation of sleep quality, evaluation of diseases of circulation system, diagnosis of sleep disorders, evaluation of effect of hypnotics, evaluation of sleep-related side effects of drugs, evaluation of influences of various regimen on sleep, evaluation of influences of various health food on sleep, evaluation of side effects of drugs on autonomic nerve function, evaluation of influences of various regimen on autonomic nerve function, evaluation of influences of various health food on autonomic nerve function, evaluation of health of the elders, and newborn sleep-disorders screen.

19. A method for sleep monitoring and evaluating using the miniature wireless apparatus of claim 1, comprising:
(a) collecting, processing, and recording a plurality of physiological signals;
(b) processing and modulating the said physiological signals to form modulated radio frequency signals;
(c) synchronously transmitting and receiving the modulated signals radio frequency signals corresponding to different physiological signals;
(d) receiving and recording the physiological signals by a remote receiver and further transferring the signals to remote monitoring stations through a network; and
(e) analyzing the physiological signals with a sleep-analyzing algorithm for sleep monitoring and evaluating by the remote receiver and the remote monitoring station.

20. The method of claim 19, wherein the physiological signals comprises an electroencephalogram signal, an electrooculogram signal, an electrocardiogram signal, an electromyogram signal, an electrogastrography signal, a temperature signal, and an acceleration signal.

21. The method of claim 19, wherein the remote receiver is a fixed or portable microcomputer system.

22. The method of claim 19, wherein the result of the analysis is further applied to medical uses comprising diagnosis of sleep disorders, evaluation of effect of hypnotics, evaluation of sleep-related side effects of drugs, evaluation of influences of various regimen on sleep, evaluation of influences of various health food on sleep, evaluation of health of the elders, and newborn sleep-disorders screen.

23. A method for monitoring and evaluating of autonomic nerve function using the miniature wireless apparatus of claim 1, comprising:
(a) collecting, processing, and recording a plurality of physiological signals;
(b) processing and modulating the said physiological signals to form modulated radio frequency signals;
(c) synchronously transmitting and receiving the modulated signals radio frequency signals corresponding to different physiological signals;
(d) receiving and recording the physiological signals by a remote receiver and further transferring the signals to remote monitoring stations through a network; and
(e) analyzing the physiological signals with an autonomic nervous-analyzing algorithm for monitoring and evaluating of autonomic nerve function by the remote receiver and the remote monitoring station.

24. The method of claim 23, wherein the physiological signals comprises an electroencephalogram signal, an electrooculogram signal, an electrocardiogram signal, an electromyogram signal, an electrogastrography signal, a temperature signal, and an acceleration signal.

25. The method of claim 23, wherein the remote receiver is a fixed or portable microcomputer system.

26. The method of claim 23, wherein the result of the analysis is further applied to medical uses comprising evaluation of side effects of drugs on autonomic nerve function, evaluation of influences of various regimen on autonomic nerve function, evaluation of influences of various health food on autonomic nerve function, and evaluation of health of the elders.

27. A method for recording and remote monitoring physiological signals, comprising:
(a) collecting one or more physiological signals by a signal collecting module;
(b) processing the said physiological signals by a signal processing module and recording the processed signals by a recording device;
(c) modulating the said processed signals, and synchronously transmitting and receiving the modulated signals from different miniature wireless apparatuses by a wireless module to integrate the physiological signals;
(d) receiving the said modulated signals by a remote receiver; and
(e) recording, monitoring, and analyzing the physiological signals by a microcomputer system of the remote receiver and further transmitting the physiological signals to remote monitoring stations through a network.

28. The method of claim 27, wherein the signal collecting module comprises wireless or non-wireless sensors.

29. The method of claim 27, wherein the signal processing module comprises an amplifier for amplifying the electrical physiological signals and a microcontroller for performing analog-to-digital conversion and data compression for an amplified physiological signal generated by the amplifier

30. The method of claim 27, wherein the wireless module comprises an RF transceiver and an antenna for modulating the digital signals from the signal processing module to form modulated radio frequency signals, transmitting the modulated radio frequency signals to an internal and a remote receiver, and receiving external wireless signals.

31. The method of claim 27, wherein the synchronously transmitting and receiving the modulated signals from different apparatuses is carried out by dividing the signal receiving time into equal intervals and corresponding each divided time-interval to each physiological signal by a firmware on the wireless module.

32. The method of claim 30, wherein the remote receiver is a fixed or portable microcomputer system.

33. The method of claim 30, wherein the external wireless signals comprises the signals of remote manipulation of the apparatus and the physiological signals from other miniature wireless apparatuses used simultaneously on the body of the user.

34. The method of claim 30, wherein the physiological signals are analyzed by the remote receiver and the remote monitoring station and are further applied to medical uses comprising evaluation of sleep quality, evaluation of diseases of circulation system, diagnosis of sleep disorders, evaluation of effect of hypnotics, evaluation of sleep-related side effects of drugs, evaluation of influences of various regimen on sleep, evaluation of influences of various health food on sleep, evaluation of side effects of drugs on autonomic nerve function, evaluation of influences of various regimen on autonomic nerve function, evaluation of influences of various health food on autonomic nerve function, evaluation of health of the elders, and newborn sleep-disorders screen.
